(19) United States
(12) Patent Application Publication
(10) Pub. No.: US 2009/0048278 A1
(43) Pub. Date: Feb. 19, 2009

(54) Novel Quinazoline-2,4-Diamine Derivatives and Their Use as Modulators of Small-Conductance Calcium-Activated Potassium Channels

(76) Inventors: Ulrik Svane Sorensen, Soborg (DK); Birgitte L. Eriksen, Farum (DK); Lene Teuber, Vaerlose (DK); Dan Peters, Malmo (SE); Dorte Strobaek, Farum (DK); Tina Holm Johansen, Smorum (DK); Palle Christophersen, Ballerup (DK)

Correspondence Address: BIRCH STEWART KOLASCH & BIRCH, PO BOX 747, FALLS CHURCH, VA, 22040-0747, US

(21) Appl. No.: 12/095,899

(22) PCT Filed: Dec. 6, 2006
(86) PCT No.: PCT/EP06/69380

§ 371 Date: Jun. 2, 2008

Related U.S. Application Data
(60) Provisional application No. 60/784,221, filed on Mar. 21, 2006.

(30) Foreign Application Priority Data
Dec. 7, 2005 (DK) PA 205 01732

(51) Int. Cl. A61K 31/517; C07D 239/95

(52) U.S. Cl. 514/266.4; 544/291

(57) ABSTRACT

This invention relates to novel quinazoline-2,4-diamine derivatives useful as modulators of small-conductance calcium-activated potassium channels (SK channels). In other aspects the invention relates to the use of these compounds in a method for therapy and to pharmaceutical compositions comprising the compounds of the invention.

### TECHNICAL FIELD

This invention relates to novel quinazoline-2,4-diamine derivatives useful as modulators of small conductance calcium-activated potassium channels (SK channels). In other aspects the invention relates to the use of these compounds in a method for therapy and to pharmaceutical compositions comprising the compounds of the invention.

### BACKGROUND ART

Three subtypes of small conductance calcium-activated potassium channels (SK channels) have been cloned: SK1, SK2 and SK3 (corresponding to K_{Ca}2.1-K_{Ca}2.3 using the IUPHAR and to KCNN1-3 using the genomic nomenclature). The activity of these channels is determined by the concentration of free intracellular calcium ([Ca²⁺]ᵢ) via calmodulin that is constitutively bound to the channels. SK channels are tightly regulated by [Ca²⁺]ᵢ in the physiological range being closed at [Ca²⁺]ᵢ up to around 0.1 μM but fully activated at a [Ca²⁺]ᵢ of 1 μM. Being selective for potassium, open (active) SK channels have an hyperpolarizing influence on the membrane potential of the cell. SK channels are widely expressed in the central nervous system. The distribution of SK1 and SK2 show a high degree of overlap and display the highest levels of expression in neocortical, limbic and hippocampal areas in the mouse brain. In contrast, the SK3 channels show high levels of expression in the basal ganglia, thalamus and the brain stem monoaminergic neurons e.g. dorsal raphe, locus coeruleus and the ventral tegmental area (Sailer et al: “Comparative immunohistochemical distribution of three small conductance Ca²⁺-activated potassium channel subunits, SK1, SK2, and SK3 in mouse brain; Mol. Cell. Neurosci. 2004 26 458-469). The SK channels are also present in several peripheral cells including skeletal muscle, gland cells, liver cells and T-lymphocytes.

The hyperpolarizing action of active SK channels plays an important role in the control of firing pattern and excitability of excitable cells. SK channel inhibitors such as apamin and quaternized derivatives of bicuculline have been demonstrated to increase excitability whereas the opener 1-EBIO is able to reduce electrical activity. In non-excitable cells where the amount of Ca²⁺ influx via voltage-independent pathways is highly sensitive to the membrane potential an activation of SK channels will increase the driving force whereas a blocker of SK channels will have a depolarizing effect and thus diminish the driving force for calcium.

Based on the important role of SK channels in linking [Ca²⁺]ᵢ and membrane potential, SK channels are an interesting target for developing novel therapeutic agents.

A review of SK channels and SK channel modulators may be found in Liegeois, J.-F. et al.: “Modulation of small conductance calcium-activated potassium (SK) channels: a new challenge in medicinal chemistry”, Current Medicinal Chemistry 2003 10 625-647.

Known modulators of SK channels suffer from being large molecules or peptides (apamin, scyllatoxin, tubocurarine, dequalinium chloride, UCL1684) or having low potency (1-EBIO, riluzole). Thus, there is a continued need for compounds with an optimized pharmacological profile. In particular, there is a great need for selective ligands, such as SK3 channel modulators.

### SUMMARY OF THE INVENTION

In its first aspect, the invention provides a compound of Formula I:

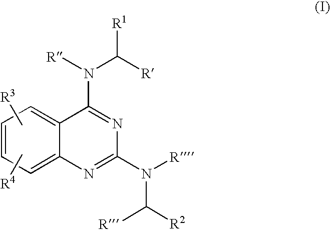

any of its isomers or any mixture of its isomers, or a pharmaceutically acceptable salt thereof, wherein

R¹ represents —(CH₂)ᵥ—R⁵; wherein v is 0 or 1 and R⁵ is a phenyl group, which phenyl group is optionally substituted with one or more substituents independently selected from the group consisting of halo, trifluoromethyl, trifluoromethoxy, cyano, alkyl, R^{e}R^{f}N— and R^{e}R^{f}N-alkyl;

R′ and R″ independent of each other are hydrogen or R^{a}-alkyl; or

R′ together with R″ form —(CH₂)ₚ—; wherein p is 3, 4 or 5; or

R′ forms a —(CH₂)_{q}— bridge to an ortho position of the phenyl group of R¹; wherein q is 2, 3 or 4; and R″ is hydrogen or R^{a}-alkyl;

R² represents —(CH₂)_{w}R⁶; wherein w is 0 or 1 and R⁶ is a phenyl group, which phenyl group is optionally substituted with one or more substituents independently selected from the group consisting of halo, trifluoromethyl, trifluoromethoxy, cyano, alkyl, R^{g}R^{h}N— and R^{g}R^{h}N-alkyl;

R′″ and R″″ independent of each other are hydrogen or R^{b}-alkyl; or

R′″ together with R″″ form —(CH₂)ₛ—; wherein s is 3, 4 or 5; or

R′″ forms a —(CH₂)ₜ— bridge to an ortho position of the phenyl group of R²; wherein t is 2, 3 or 4; and R″″ is hydrogen or R^{b}-alkyl;

R^{a} and R^{b} independent of each other represent hydrogen, hydroxy, cyano, or R^{c}R^{d}N—; wherein R^{c} and R^{d} independent of each other represent hydrogen or alkyl;

R^{e}, R^{f}, R^{g} and R^{h} independent of each other are hydrogen or alkyl; and

R³ and R⁴ independent of each other are selected from the group consisting of hydrogen, halo, trifluoromethyl, trifluoromethoxy, cyano, alkyl and alkoxy.

In its second aspect, the invention provides a pharmaceutical composition, comprising a therapeutically effective amount of a compound of the invention, any of its isomers or any mixture of its isomers, or a pharmaceutically acceptable salt thereof, together with at least one pharmaceutically acceptable carrier, excipient or diluent.

In a further aspect, the invention provides the use of a compound of the invention, any of its isomers or any mixture of its isomers, or a pharmaceutically acceptable salt thereof for the manufacture of a pharmaceutical composition for the treatment, prevention or alleviation of a disease or a disorder or a condition of a mammal, including a human, which disease, disorder or condition is responsive to modulation of SK channels.

In a still further aspect, the invention relates to a method for treatment, prevention or alleviation of a disease or a disorder or a condition of a living animal body, including a human, which disorder, disease or condition is responsive to modulation of SK channels, which method comprises the step of administering to such a living animal body in need thereof a therapeutically effective amount of a compound of the invention, any of its isomers or any mixture of its isomers, or a pharmaceutically acceptable salt thereof.

Other objects of the invention will be apparent to the person skilled in the art from the following detailed description and examples.

### DETAILED DISCLOSURE OF THE INVENTION

### Quinazoline-2,4-diamine derivatives

In its first aspect the present invention provides a quinazoline-2,4-diamine derivative of Formula I:

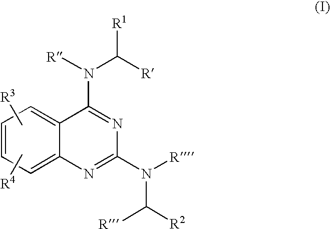

any of its isomers or any mixture of its isomers, or a pharmaceutically acceptable salt thereof wherein

R¹ represents —(CH₂)ᵥ—R⁵; wherein v is 0 or 1 and R⁵ is a phenyl group, which phenyl group is optionally substituted with one or more substituents independently selected from the group consisting of halo, trifluoromethyl, trifluoromethoxy, cyano, alkyl, R^{e}R^{f}N— and R^{e}R^{f}N-alkyl;

R′ and R″ independent of each other are hydrogen or R^{a}-alkyl; or

R′ together with R″ form —(CH₂)ₚ—; wherein p is 3, 4 or 5; or

R′ forms a —(CH₂)_{q}— bridge to an ortho position of the phenyl group of R¹; wherein q is 2, 3 or 4; and R″ is hydrogen or R^{a}-alkyl;

R² represents —(CH₂)_{w}R⁶; wherein w is 0 or 1 and R⁶ is a phenyl group, which phenyl group is optionally substituted with one or more substituents independently selected from the group consisting of halo, trifluoromethyl, trifluoromethoxy, cyano, alkyl, R^{g}R^{h}N— and R^{g}R^{h}N-alkyl;

R′″ and R″″ independent of each other are hydrogen or R^{b}-alkyl; or

R′″ together with R″″ form —(CH₂)ₛ—; wherein s is 3, 4 or 5; or

R′″ forms a —(CH₂)ₜ— bridge to an ortho position of the phenyl group of R²; wherein t is 2, 3 or 4; and R″″ is hydrogen or R^{b}-alkyl;

R^{a} and R^{b} independent of each other represent hydrogen, hydroxy, cyano, or R^{c}R^{d}N—; wherein R^{c} and R^{d} independent of each other represent hydrogen or alkyl;

R^{e}, R^{f}, R^{g} and R^{h} independent of each other are hydrogen or alkyl; and

R³ and R⁴ independent of each other are selected from the group consisting of hydrogen, halo, trifluoromethyl, trifluoromethoxy, cyano, alkyl and alkoxy.

In a preferred embodiment the quinazoline-2,4-diamine derivative of the invention is a compound of Formula I, wherein R¹ represents —(CH₂) R⁵; wherein v is 0 or 1 and R⁵ is a phenyl group, which phenyl group is optionally substituted with one or more substituents independently selected from the group consisting of halo, trifluoromethyl, trifluoromethoxy, cyano, alkyl, R^{e}R^{f}N— and R^{e}R^{f}N-alkyl;

R′ and R″ independent of each other are hydrogen or R^{a}-alkyl; or

R′ together with R″ form —(CH₂)ₚ—; wherein p is 3, 4 or 5; or

R′ forms a —(CH₂)_{q}— bridge to an ortho position of the phenyl group of R¹; wherein q is 2, 3 or 4; and R⁷ is hydrogen or R^{a}-alkyl;

wherein R^{e} and R^{f} are as defined above.

In a more preferred embodiment R¹ represents —(CH₂)ᵥ—R⁵; wherein v is 0, and R⁵ is a phenyl group, which phenyl group is optionally substituted with one or two substituents independently selected from the group consisting of halo, trifluoromethyl and N,N-dimethyl-amino; and

R′ and R″ both represent hydrogen.

In an even more preferred embodiment R¹ represents —(CH₂)ᵥR⁵; wherein v is 0, and R⁵ is a phenyl group, which phenyl group is optionally substituted with one or two substituents independently selected from the group consisting of fluoro, chloro, bromo, trifluoromethyl and N,N-dimethyl-amino; and

R′ and R″ both represent hydrogen.

In a still more preferred embodiment R¹ represents —(CH₂)ᵥR⁵; wherein v is 0, and R⁵ is a phenyl group, which phenyl group is optionally substituted with one or two substituents independently selected from the group consisting of halo, trifluoromethyl and N,N-dimethyl-amino; and

R′ forms a —(CH₂)_{q}— bridge to an ortho position of the phenyl group of R¹; wherein q is 2, 3 or 4; and R″ is hydrogen.

In a yet more preferred embodiment R¹ represents —(CH₂)ᵥ—R⁵; wherein v is 0, and R⁵ is a phenyl group, which phenyl group is optionally substituted with one or two substituents independently selected from the group consisting of halo, trifluoromethyl and N,N-dimethyl-amino; and

R′ forms a —(CH₂)_{q}— bridge to an ortho position of the phenyl group of R¹; wherein q is 3; and R″ is hydrogen.

In a further more preferred embodiment R¹ represents —(CH₂)ᵥ—R⁵; wherein v is 0, and R⁵ is a phenyl group, which phenyl group is optionally substituted with one or two substituents independently selected from the group consisting of fluoro, chloro, bromo, trifluoromethyl and N,N-dimethyl-amino; and

R′ forms a —(CH₂)_{q}— bridge to an ortho position of the phenyl group of R¹; wherein q is 3; and R″ is hydrogen.

In a preferred embodiment the quinazoline-2,4-diamine derivative of the invention is a compound of Formula I, wherein R¹ represents

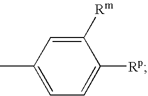

wherein R^{m} and R^{p} independent of each other are selected from the group consisting of hydrogen, halo, trifluoromethyl, trifluoromethoxy, cyano, alkyl, R^{e}R^{f}N— and R^{e}R^{f}—N-alkyl; and wherein R^{e} and R^{f} are as defined above.

In a more preferred embodiment R¹ represents

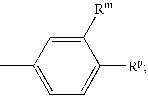

wherein R^{m} and R^{p} independent of each other are selected from the group consisting of hydrogen, fluoro, chloro, bromo, trifluoromethyl and N,N-dimethyl-amino.

In an even more preferred embodiment R^{m} represents hydrogen or fluoro, and R^{p} represents fluoro, chloro or N,N-dimethyl-amino.

In a still more preferred embodiment R^{m} represents hydrogen; and R^{p} represents chloro or N,N-dimethyl-amino.

In a yet more preferred embodiment R^{m} and R^{p} both represent fluoro.

In a further more preferred embodiment R^{m} and R^{p} both represent hydrogen.

In a preferred embodiment the quinazoline-2,4-diamine derivative of the invention is a compound of Formula I, wherein

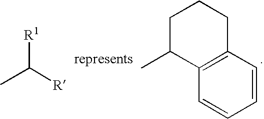

In a preferred embodiment the quinazoline-2,4-diamine derivative of the invention is a compound of Formula II, wherein R² represents

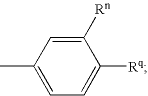

wherein Rⁿ and R^{p} independent of each other are selected from the group consisting of hydrogen, halo, trifluoromethyl, trifluoromethoxy, cyano, alkyl, R^{g}R^{h}N— and R^{g}R^{h}—N-alkyl; and wherein R^{g} and R^{h} are as defined above.

In a more preferred embodiment Rⁿ and R^{q} independent of each other are selected from the group consisting of hydrogen, halo, trifluoromethyl and R^{g}R^{h}—N-alkyl; and wherein R⁹ and R^{h} both represent hydrogen.

In an even more preferred embodiment Rⁿ and R^{q} independent of each other are selected from the group consisting of hydrogen, fluoro, chloro and H₂N-methyl.

In a still more preferred embodiment Rⁿ represents hydrogen, fluoro or H₂N-methyl; and R^{q} represents hydrogen, chloro or fluoro.

In a preferred embodiment the quinazoline-2,4-diamine derivative of the invention is a compound of Formula I, wherein

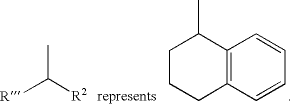

In a preferred embodiment the quinazoline-2,4-diamine derivative of the invention is a compound of Formula I, wherein

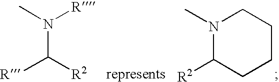

wherein R² is as defined above.

In a preferred embodiment the quinazoline-2,4-diamine derivative of the invention is a compound of Formula I, wherein v is 0 and w is 0.

In a most preferred embodiment the quinazoline-2,4-diamine derivative of the invention is
N²,N⁴-Bis(4-chlorobenzyl)quinazoline-2,4-diamine;
N²,N⁴-Bis(3,4-difluorobenzyl)quinazoline-2,4-diamine;
N²-(3-Aminomethylbenzyl)-N⁴-(3,4-difluorobenzyl)quinazoline-2,4-diamine;
N²-[1-(4-Fluorophenyl)ethyl)]-N⁴-(3,4-difluorobenzyl)quinazoline-2,4-diamine;
N²-(3,4-Difluorobenzyl)-N-(4-dimethylaminobenzyl quinazoline-2,4-diamine; (4-Chlorobenzyl)-[2-(2-phenylpiperidin-1-yl)quinazolin-4-yl]amine;
N²-Benzyl-N⁴-(4-chlorobenzyl)-N²-(hydroxyethyl)quinazoline-2,4-diamine;
N²-Benzyl-N⁴-(4-chlorobenzyl)-N²-(cyanoethyl)quinazoline-2,4-diamine;
N²-Benzyl-N⁴-(4-chlorobenzyl)-N²-(dimethylaminoethyl)quinazoline-2,4-diamine;
N⁴-(1,2,3,4-Tetrahydronaphthalen-1-yl)-N²—(R)-(1,2,3,4-tetrahydronaphthalen-1-yl)quinazoline-2,4-diamine;
N²-(4-Chlorobenzyl)-N⁴-(1,2,3,4-tetrahydronaphthalen-1-yl)quinazoline-2,4-diamine;
N⁴-(4-Chlorobenzyl)-N²—(R)-(1,2,3,4-tetrahydronaphthalen-1-yl)quinazoline-2,4-diamine;

or a pharmaceutically acceptable salt thereof.

Any combination of two or more of the embodiments as described above is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contains of from one to six carbon atoms (C₁₋₆-alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

Alkoxy is O-alkyl, wherein alkyl is as defined above.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the compounds of the present invention may contain one or more chiral centers, and that such compounds exist in the form of isomers.

Moreover, the chemical compounds of the present invention may exist as enantiomers in (+) and (−) forms as well as in racemic forms (±). The racemates of these isomers and the individual isomers themselves are within the scope of the present invention.

The invention includes all such isomers and any mixtures thereof including racemic mixtures.

Racemic forms can be resolved into the optical antipodes by known methods and techniques. One way of separating the isomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l-(tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radio-nuclide as a label. Positron emitting radionuclides are all candidates for usage. In the context of this invention the radionuclide is preferably selected from ²H (deuterium), ³H (tritium), ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention may be tested for their ability to modulate SK channels in vitro. Functional modulation can be determined by measuring the compound-induced change in SK current by the patch clamp technique as described in Strøbaek et al.: “Pharmacological characterization of small-conductance Ca²⁺-activated K channels expressed in HEK293 cells”, British Journal of Pharmacology 2000 129 991-999. From this type of measurements the potency of a given compound can be determined as e.g. Kᵢ or IC₅₀ values for blockers/inhibitors and EC₅₀ values for openers/activators. Similar data can be obtained from other patch clamp configurations and from channels expressed endogenously in various cell lines.

In one embodiment, the compounds of the invention show selectivity for SK3 over SK1 and SK2. In a further embodiment, the compounds of the invention are positive SK channel modulators, such as positive SK3 channel modulators. In a still further embodiment, the compounds of the invention are negative modulators, such as negative SK3 channel modulators. In a special embodiment, the compounds of the invention are SK channel blockers, such as SK3 channel blockers.

Based on the activity observed in the patch clamp experiments, the compound of the invention is considered useful for the treatment, prevention or alleviation of a disease or a disorder or a condition of a mammal, including a human, which disease, disorder or condition is responsive to modulation of SK channels.

In a special embodiment, the compounds of the invention are considered useful for the treatment, prevention or alleviation of: absence seizures, agerelated memory loss, Alzheimer's disease, angina pectoris, arrhythmia, asthma, anxiety, ataxia, attention deficits, baldness, bipolar disorder, bladder hyperexcitability, bladder outflow obstruction, bladder spasms, brain tumors, cerebral ischaemia, chronic obstructive pulmonary disease, cancer, cardiovascular disorders, cognitive dysfunction, colitis, constipation, convulsions, coronary artery spasms, coronary hearth disease, cystic fibrosis, dementia, depression, diabetes type II, dysmenorrhoea, epilepsy, gastrointestinal dysfunction, gastroesophageal reflux disorder, gastrointestinal hypomotility disorders gastrointestinal motility insufficiency, hearing loss, hyperinsulinemia, hypertension, immune suppression, inflammatory bowel disease, inflammatory pain, intermittent claudication, irritable bowel syndrome, ischaemia, ischaemic hearth disease, learning deficiencies, male erectile dysfunction, manic depression, memory deficits, migraine, mood disorders, motor neuron diseases, myokymia, myotonic dystrophy, myotonic muscle dystrophia, narcolepsy, neuropathic pain, pain, Parkinson's disease, polycystic kidney disease, postoperative ileus, premature labour, psychosis, psychotic disorders, renal disorders, Reynaud's disease, rhinorrhoea, secretory diarrhoea, seizures, Sjorgren's syndrome, sleep apnea, spasticity, sleeping disorders, stroke, traumatic brain injury, trigeminal neuralgia, urinary incontinence, urinogenital disorders; vascular spasms, vision loss, and xerostomia.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drags, in powder, or in liquid form, and parenteral administration, in particular cutaneous, subcutaneous, intramuscular, or intravenous injection. The pharmaceutical composition of the invention can be prepared by any skilled person by use of standard methods and conventional techniques appropriate to the desired formulation. When desired, compositions adapted to give sustained release of the active ingredient may be employed.

Pharmaceutical compositions of the invention may be those suitable for oral, rectal, bronchia, nasal, pulmonal, topical (including buccal and sub-lingual, transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose a low melting wax, cocoa butter; and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets; capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to modulation of SK channels, and which method comprises administering to such a living animal body, including a human, in need thereof an effective amount of a chemical compound of the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The invention is further illustrated with reference to the following examples, which are not intended to be in any way limiting to the scope of the invention as claimed.

General: The procedures represent generic procedures used to prepare compounds of the invention. Abbreviations used are as follows:

### Ac: Acetyl

### Et: Ethyl

Eq: equivalents
LCMS: Liquid chromatography mass spectrometry
Me: methyl
mp: melting point
MW: microwave
rt: room temperature
TEA: triethylamine

### Procedure A

2,4-Dichloroquinazoline and two eq of the required amine were suspended in acetonitrile in a closed vial and heated to 150-200° C. for 15-45 min by use of MW irradiation. After cooling to rt the precipitated solid was filtered off and purified by column chromatography or preparative LCMS to give the desired product as the free base. Alternatively, this product was isolated as a HCl salt upon filtration of the reaction mixture and recrystallization.

An example of Procedure A, the preparation of N²,N⁴-bis(3,4-difluorobenzyl)-quinazoline-2,4-diamine, is shown in Scheme 1.

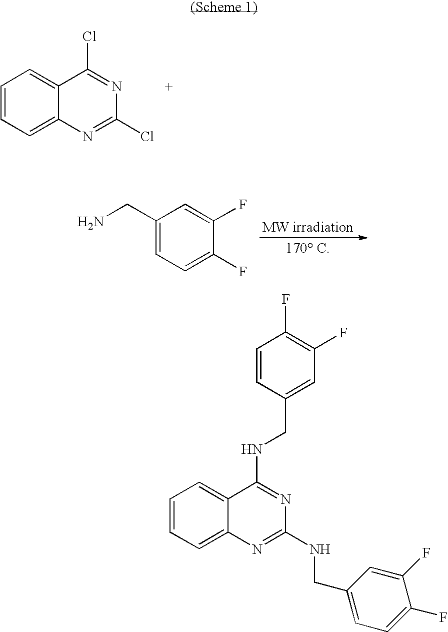

### Procedure B

2,4-Dichloroquinazoline, TEA (5 eq) and 1.1 eq of an aryl amine (amine A, Scheme 2) were suspended in acetonitrile and stirred at rt overnight. Water was added and the mixture extracted with EtOAc. The combined organic phases were dried (MgSO₄), filtered and concentrated in vacuo to give the crude 4-arylalkylamino-2-chloroquinazoline in high yield. Subsequently, this intermediate was dissolved in acetonitrile, added 1.2 eq of the second aryl amine (amine B, Scheme 2) and the reaction mixture heated to 150-200° C. for 15-80 min by use of MW irradiation.

Alternatively, the crude reaction mixture after step 1 was used without isolation of the 4-arylalkylamino-2-chloroquinazoline, added amine B and heated in the MW oven.

After step 2, the crude product was isolated by aqueous work-up as described above, or by filtration from the reaction mixture, and subsequently purified by column chromatography or preparative LCMS to yield the desired 2,4-bis-(arylalkylamino)-quinazoline as the free base. Alternatively, this product was isolated as a HCl salt upon filtration of the reaction mixture and recrystallization.

An example of Procedure B, the preparation of N²-[1-(4-fluorophenyl)ethyl)]-N⁴-(3,4-difluorobenzyl)quinazoline-2,4-diamine, is shown in Scheme 2.

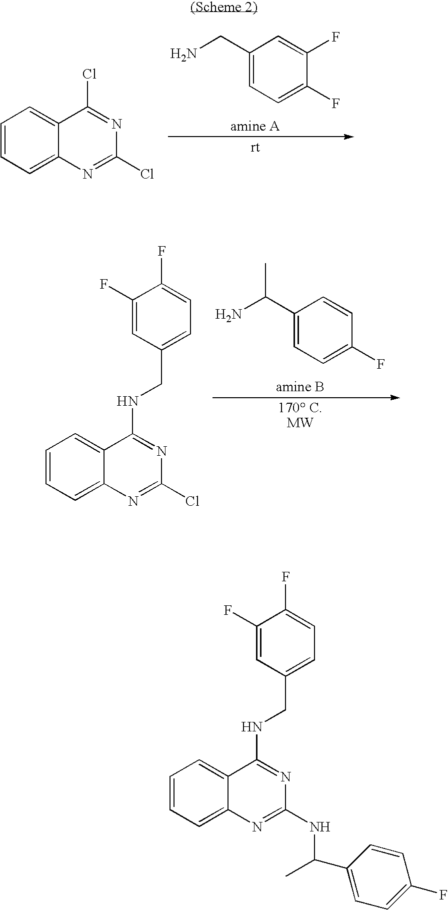

### Example 1

### N²,N⁴-Bis(4-chlorobenzyl)quinazoline-2,4-diamine

The title compound was prepared in two steps from 2,4-dichloroquinazoline and 4-chlorobenzylamine by Procedure B. After reacting (4-chlorobenzyl)-(2-chloroquinazoline-4-yl)amine with 4-chlorobenzylamine, the title compound was isolated by filtration, refluxed in MeOH, cooled and isolated by filtration as the HCl salt (mp 263-264° C.). MS (ES⁺) m/z 409 (M⁺, 100).

### Example 2

### N²,N⁴-Bis(3,4-difluorobenzyl)quinazoline-2,4-diamine

The title compound was prepared in one step from 2,4-dichloroquinazoline and 3,4-difluorobenzylamine by Procedure A. The precipitated solid was filtered off and washed with acetonitrile to give the title compound as the HCl salt (mp 275-276° C.).

MS (ES⁺) m/z 413 ([M+1]⁺, 100).

### Example 3

### N²-(3-Aminomethylbenzyl)-N⁴-(3,4-difluorobenzyl)quinazoline-2,4-diamine

The title compound was prepared by Procedure B in two steps from 2,4-dichloro-quinazoline, 3,4-difluorobenzylamine and, in the second step, m-xylylenediamine. Upon aqueous work-up, the crude product was purified by preparative LCMS to give the title compound as the free base (solid, mp 148-149° C.). MS (ES⁺) m/z 406 ([M+1]⁺, 100).

### Example 4

### N²-[1-(4-Fluorophenyl)ethyl)]-N⁴-(3,4-difluorobenzyl)quinazoline-2,4-diamine

The title compound was prepared by Procedure B in two steps from 2,4-dichloro-quinazoline, 3,4-difluorobenzylamine and, in the second step, 1-(4-fluorophenyl)-ethylamine. Upon aqueous work-up, the crude product was purified by preparative LCMS to give the title compound as the free base (solid, mp 58-59° C.). MS (ES⁺) m/z 409 ([M+1]⁺, 100).

### Example 5

### N²-(3,4-Difluorobenzyl)-N⁴-(4-dimethylaminobenzyl)quinazoline-2,4-diamine

The title compound was prepared by Procedure B in two steps from 2,4-dichloro-quinazoline, 4-(dimethylamino)benzylamine and, in the second step, 3,4-difluoro-benzylamine. Upon aqueous work-up, the crude product was purified by preparative LCMS to give the title compound as the free base (solid, mp 59-60° C.). MS (ES⁺) m/z 420 ([M+1]⁺, 100).

### Example 6

### (4-Chlorobenzyl)-[2-(2-phenylpiperidin-1-yl)quinazolin-4-yl]amine

The title compound was prepared by Procedure B in two steps from 2,4-dichloro-quinazoline, 4-chlorobenzylamine and, in the second step, 2-phenylpiperidine (Healy, Maureen A. M.; Smith, Stephen A.; Stemp, Geoffrey, Synth. Commun. (1995), 25, 3789-97). Upon aqueous work-up, the crude product was purified by preparative LCMS to give the title compound as the free base (solid, mp 86-87° C.). MS (ES⁺) m/z 429 (M⁺, 100).

### Example 7

### N²-Benzyl-N⁴-(4-chlorobenzyl)-N²-(hydroxyethyl)quinazoline-2,4-diamine

The title compound was prepared by Procedure B in two steps from 2,4-dichloro-quinazoline, 4-chlorobenzylamine and, in the second step, N-benzylethanolamine. The product was isolated by filtration and subsequent purification by preparative LCMS to give the title compound as the free base (solid, mp 188-190.5° C.). MS (ES⁺) m/z 419 (M⁺, 100).

### Example 8

### N²-Benzyl-N⁴-(4-chlorobenzyl)-N²-(cyanoethyl)quinazoline-2,4-diamine

The title compound was prepared by Procedure B in two steps from 2,4-dichloro-quinazoline, 4-chlorobenzylamine and, in the second step, 3-(benzylamino)-propionitrile. Upon aqueous work-up, the crude product was purified by preparative LCMS to give the title compound as the free base (oil). MS (ES⁺) m/z 428 (M⁺, 100). ¹H NMR (CDCl₃) δ 2.68 (br s, 2H), 3.88 (br s, 2H), 4.70 (s, 2H), 5.02 (s, 2H), 6.10 (br s, 1H), 7.02-7.38 (m, 10H), 7.50-7.63 (m, 3H).

### Example 9

### N²-Benzyl-N⁴-(4-chlorobenzyl)-N²-(dimethylaminoethyl)quinazoline-2,4-diamine

The title compound was prepared by Procedure B in two steps from 2,4-dichloro-quinazoline, 4-chlorobenzylamine and, in the second step, N′-benzyl-N,N-dimethyl-ethylenediamine. Upon aqueous work-up, the crude product was purified by preparative LCMS to give the title compound as the free base (oil). MS (ES⁺) m/z 446 (M⁺, 100). ¹H NMR (CDCl₃) δ 2.25 (br s, 6H), 2.55 (br s, 2H), 3.76 (br s, 2H), 4.72 (br s, 2H), 5.00 (br s, 2H), 6.00 (br s, 1H), 7.02-7.40 (m, 10H), 7.48-7.60 (m, 3H).

### Example 10

### N⁴-(1,2,3,4-Tetrahydronaphthalen-1-yl)-N²—(R)-(1,2,3,4-tetrahydronaphthalen-1-yl)quinazoline-2,4-diamine

The title compound was prepared by Procedure B in two steps from 2,4-dichloro-quinazoline, 1,2,3,4-tetrahydro-1-naphtylamine and, in the second step, (R)-1,2,3,4-tetrahydro-1-naphtylamine. Upon aqueous work-up, the crude product was purified by column chromatography (EtOAc/heptane/TEA, 1:2, 1%) to give the title compound as the free base (solid, mp 96-97° C.). MS (ES⁺) m/z 421 ([M+1]⁺, 100).

### Example 11

### N²-(4-Chlorobenzyl)-N⁴-(1,2,3,4-tetrahydronaphthalen-1-yl)quinazoline-2,4-diamine

The title compound was prepared by Procedure B in two steps from 2,4-dichloro-quinazoline, 1,2,3,4-tetrahydro-1-naphtylamine and, in the second step, 4-chloro-benzylamine. The precipitated solid was filtered off, washed with acetonitrile and recrystallized from MeOH to give the title compound as the HCl salt (mp 166-167° C.). MS (ES⁺) m/z 415 (M⁺, 100).

### Example 12

### N⁴-(4-Chlorobenzyl)-N²—(R)-(1,2,3,4-tetrahydronaphthalen-1-yl)quinazoline-2,4-diamine

The title compound was prepared by Procedure B in two steps from 2,4-dichloro-quinazoline, 4-chlorobenzylamine and, in the second step, (R)-1,2,3,4-tetrahydro-1-naphtylamine. Upon aqueous work-up, the title product was isolated without further purification as the free base (solid, mp 85-86° C.). MS (ES⁺) m/z 415 (M⁺, 100).

### Example 13

### Biological Activity

This example demonstrates the biological activity of a compound representative of the invention, i.e. the compound of Example 8. The ionic current through small-conductance Ca²⁺-activated K⁺ channels (SK channels, subtype 3) was recorded using the whole-cell configuration of the patch-clamp technique.

HEK293 tissue culture cells expressing hSK3 channels were grown in DMEM (Dulbecco's Modified Eagle Medium) supplemented with 10% FCS (foetal calf serum) at 37° C. in 5% CO₂. At 60-80% confluency, cells were harvested by trypsin treatment and seeded on cover slips.

Cells plated on coverslips are placed in a 15 μl perfusion chamber (flowrate ˜1 ml/min) mounted on an inverted microscope placed on a vibration-free table in a grounded Faraday cage. The experiments were performed at room temperature (20-22° C.). The EPC-9 patch-clamp amplifier (HEKA-electronics, Lambrect, Germany) was connected to a Macintosh computer via an ITC16 interface. Data were stored directly on the hard-disk and analysed by IGOR software (Wavemetrics, Lake Oswega, Oreg., USA).

The whole-cell configuration of the patch-clamp technique was applied. In short: The tip of a borosilicate pipette (resistance 2-4 MΩ) is gently placed on the cell membrane using remote control systems. Light suction results in the formation of a giga seal (pipette resistance increases to more than 1 GΩ) and the cell membrane underneath the pipette is then ruptured by more powerful suction. Cell capacitance was electronically compensated and the resistance between the pipette and the cell interior (the series resistance, R⁵) was measured and compensated for. The cell capacitances ranged from 5 to 20 pF, and the series resistance was in the range 3 to 6 MΩ. Rs- as well as capacitance compensation were updated during the experiments (before each stimulus). Leak-subtractions were not performed.

The extracellular (bath) solution contained (in mM): 156 KCl, 0.1 CaCl₂, 3 MgCl₂, 10 HEPES (pH=7.4 with KOH). The test compound was dissolved in DMSO and then diluted at least 1000 times in the extracellular solution.

The intracellular (pipette) solution contained: 154 mM KCl, 10 mM HEPES, 10 mM EGTA. Concentrations of CaCl₂ and MgCl₂ needed to obtain the desired free concentrations of Ca²⁺ (0.3-0.4 μM, Mg²⁺ always 1 mM) were calculated by EqCal software (Cambridge, UK) and added.

After establishment of the whole-cell configuration, voltage-ramps (−80 to +80 mV) were applied to the cell every 5 seconds from a holding potential of 0 mV. A stable baseline current was obtained within a period of 100-500 seconds, and the compound was then added by changing to an extracellular solution containing the test compound. Activity was quantified from the change in current at −75 mV. For inhibitors a Ki value, defined as the concentration required for decreasing the baseline current to 50% of the initial current, was estimated.

The Kᵢ value determined for the compound of Example 8 of the invention is 0.3 μM, respectively, indicating its SK3 inhibiting property.

1-14. (canceled)

15. A quinazoline-2,4-diamine derivative of Formula I: 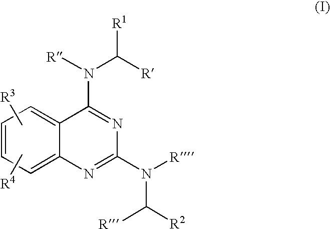
any of its stereoisomers or any mixture of its stereoisomers, or a pharmaceutically acceptable salt thereof; wherein
R¹ represents —(CH₂)ᵥ—R⁵; wherein v is 0 or 1 and R⁵ is a phenyl group;
which phenyl group is optionally substituted with one or more substituents independently selected from the group consisting of halo, trifluoromethyl, trifluoromethoxy, cyano, alkyl, R^{e}R^{f}N— and R^{e}R^{f}N-alkyl;
R′ and R″ independent of each other are hydrogen or R^{a}-alkyl; or
R′ together with R″ form —(CH₂)ₚ—; wherein p is 3, 4 or 5; or
R′ forms a —(CH₂)_{q}— bridge to an ortho position of the phenyl group of R¹; wherein q is 2, 3 or 4; and R″ is hydrogen or R^{a}-alkyl;
R² represents —(CH₂)_{w}—R⁶; wherein w is 0 or 1 and R⁶ is a phenyl group;
which phenyl group is optionally substituted with one or more substituents independently selected from the group consisting of halo, trifluoromethyl, trifluoromethoxy, cyano, alkyl, R^{g}R^{h}N— and R^{g}R^{h}N-alkyl;
R′″ and R″″ independent of each other are hydrogen or R^{b}-alkyl; or
R′″ together with R″″ form —(CH₂)ₛ—; wherein s is 3, 4 or 5; or
R′″ forms a —(CH₂)ₜ— bridge to an ortho position of the phenyl group of R²; wherein t is 2, 3 or 4; and R″″ is hydrogen or R^{b}-alkyl;
R^{a} and R^{b} independent of each other represent hydrogen, hydroxy, cyano, or R^{c}R^{d}N—; wherein R^{c} and R^{d} independent of each other represent hydrogen or alkyl;
R^{e}, R^{f}, R^{g} and R^{h} independent of each other are hydrogen or alkyl; and
R³ and R⁴ independent of each other are selected from the group consisting of hydrogen, halo, trifluoromethyl, trifluoromethoxy, cyano, alkyl and alkoxy.

16. The quinazoline-2,4-diamine derivative of claim 15, or a pharmaceutically acceptable salt thereof, wherein R¹ represents —(CH₂)ᵥ—R⁵; wherein v is 0 or 1 and R⁵ is a phenyl group, which phenyl group is optionally substituted with one or more substituents independently selected from the group consisting of halo, trifluoromethyl, trifluoromethoxy, cyano, alkyl, R^{e}R^{f}N— and R^{e}R^{f}N-alkyl;
R′ and R″ independent of each other are hydrogen or R^{a}-alkyl; or
R′ together with R″ form —(CH₂)ₚ—; wherein p is 3, 4 or 5; or
R′ forms a —(CH₂)_{q}— bridge to an ortho position of the phenyl group of R¹; wherein q is 2, 3 or 4; and R″ is hydrogen or R^{a}-alkyl;
wherein R^{e} and R^{f} are as defined in claim 15.

17. The quinazoline-2,4-diamine derivative of claim 15, or a pharmaceutically acceptable salt thereof, wherein R¹ represents 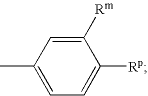
wherein R^{m} and R^{p} independent of each other are selected from the group consisting of hydrogen, halo, trifluoromethyl, trifluoromethoxy, cyano, alkyl, R^{e}R^{f}N— and R^{e}R^{f}—N-alkyl; and
wherein R^{e} and R^{f} are as defined in claim 15.

18. The quinazoline-2,4-diamine derivative of claim 15, or a pharmaceutically acceptable salt thereof, wherein

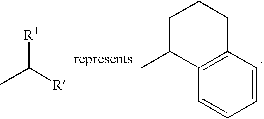

19. The quinazoline-2,4-diamine derivative of claim 15, or a pharmaceutically acceptable salt thereof wherein R² represents 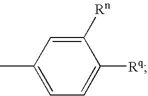
wherein Rⁿ and R^{q} independent of each other are selected from the group consisting of hydrogen, halo, trifluoromethyl, trifluoromethoxy, cyano, alkyl, R^{g}R^{h}N— and R^{g}R^{h}—N-alkyl- and
wherein R^{g} and R^{h} are as defined in claim 15.

20. The quinazoline-2,4-diamine derivative of claim 15, or a pharmaceutically acceptable salt thereof, wherein

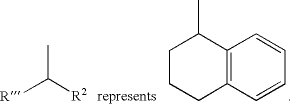

21. The quinazoline-2,4-diamine derivative of claim 15, or a pharmaceutically acceptable salt thereof, wherein 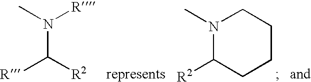
wherein R² is as defined in claim 15.

22. The quinazoline-2,4-diamine derivative of claim 15, or a pharmaceutically acceptable salt thereof, wherein v is 0 and w is 0.

23. The quinazoline-2,4-diamine derivative of claim 15, which is
N²,N⁴-Bis(4-chlorobenzyl)quinazoline-2,4-diamine;
N²,N⁴-Bis(3,4-difluorobenzyl)quinazoline-2,4-diamine;
N²-(3-Aminomethylbenzyl)-N⁴-(3,4-difluorobenzyl)quinazoline-2,4-diamine;
N²-[1-(4-Fluorophenyl)ethyl)]-M-(3,4-difluorobenzyl)quinazoline-2,4-diamine;
N²-(3,4-Difluorobenzyl)-N-(4-dimethylaminobenzyl)quinazoline-2,4-diamine;
(4-Chlorobenzyl)-[2-(2-phenylpiperidin-1-yl)quinazolin-4-yl]amine;
N²-Benzyl-N⁴-(4-chlorobenzyl)-N²-(hydroxyethyl)quinazoline-2,4-diamine;
N²-Benzyl-N⁴-(4-chlorobenzyl)-N²-(cyanoethyl)quinazoline-2,4-diamine;
N²-Benzyl-N⁴-(4-chlorobenzyl)-N²-(dimethylaminoethyl)quinazoline-2,4-diamine;
N⁴-(1,2,3,4-Tetrahydronaphthalen-1-yl)-N²—(R)-(1,2,3,4-tetrahydronaphthalen-1-yl)quinazoline-2,4-diamine;
N²-(4-Chlorobenzyl)-N⁴-(1,2,3,4-tetrahydronaphthalen-1-yl)quinazoline-2,4-diamine;
N⁴-(4-Chlorobenzyl)-N²—(R)-(1,2,3,4-tetrahydronaphthalen-1-yl)quinazoline-2,4-diamine;
or a pharmaceutically acceptable salt thereof.

24. A pharmaceutical composition, comprising a therapeutically effective amount of the quinazoline-2,4-diamine derivative of claim 15 or any of its stereoisomers or any mixture of its stereoisomers, or a pharmaceutically acceptable salt thereof, together with at least one pharmaceutically acceptable carrier, excipient or diluent.

25. The method according to claim 26, wherein the disease, disorder or condition responsive to modulation of SK channels is; absence seizures, agerelated memory loss, Alzheimer's disease, angina pectoris, arrhythmia, asthma, anxiety, ataxia, attention deficits, baldness, bipolar disorder, bladder hyperexcitability, bladder outflow obstruction, bladder spasms, brain tumors, cerebral ischaemia, chronic obstructive pulmonary disease, cancer, cardiovascular disorders, cognitive dysfunction, colitis, constipation, convulsions, coronary artery spasms, coronary hearth disease, cystic fibrosis, dementia, depression, diabetes type II, dysmenorrhoea, epilepsy, gastro-intestinal dysfunction, gastroesophageal reflux disorder, gastrointestinal hypomotility disorders gastrointestinal motility insufficiency, hearing loss, hyperinsulinemia, hypertension, immune suppression, inflammatory bowel disease, inflammatory pain, intermittent claudication, irritable bowel syndrome, ischaemia, ischaemic hearth disease, learning deficiencies, male erectile dysfunction, manic depression, memory deficits, migraine, mood disorders, motor neuron diseases, myokymia, myotonic dystrophy, myotonic muscle dystrophia, narcolepsy, neuropathic pain, pain, Parkinson's disease, polycystic kidney disease, postoperative ileus, premature labour, psychosis, psychotic disorders, renal disorders, Reynaud's disease, rhinorrhoea, secretory diarrhoea, seizures, Sjorgren's syndrome, sleep apnea, spasticity, sleeping disorders, stroke, traumatic brain injury, trigeminal neuralgia, urinary incontinence, urinogenital disorders, vascular spasms, vision loss, or xerostomia.

26. A method for treatment, prevention or alleviation of a disease or a disorder or a condition of a living animal body, including a human, which disorder, disease or condition is responsive to modulation of SK channels, which method comprises the step of administering to such a living animal body in need thereof a therapeutically effective amount of the quinazoline-2,4-diamine derivative according to claim 15, or any of its stereoisomers or any mixture of its stereoisomers, or a pharmaceutically acceptable salt thereof.
